(19) United States
(12) Patent Application Publication
(10) Pub. No.: US 2010/0209482 A1
(43) Pub. Date: Aug. 19, 2010

(54) DRESSING SECURING SYSTEM

(75) Inventor: Annette Louise Cordell, Beaumont, CA (US)

Correspondence Address: Nixon Peabody LLP, P.O. Box 60610, Palo Alto, CA, 94306, US

(73) Assignee: ACIN Pty Ltd, Beaumont, CA (US)

(21) Appl. No.: 12/597,909

(22) PCT Filed: Apr. 27, 2007
(86) PCT No.: PCT/AU07/00556

§ 371 Date: Mar. 29, 2010

(30) Foreign Application Priority Data
Apr. 27, 2006 (AU) 2006902179

(51) Int. Cl. A61K 9/70; A61P 17/02; A61F 13/00

(52) U.S. Cl. 424/445; 602/79

(57) ABSTRACT

A dressing securing system for securing a dressing relative to a wound, the dressing securing system comprising a fabric composed of a resilient knitted material.

### CROSS REFERENCE TO RELATED APPLICATIONS OR PRIORITY CLAIM

This application is a national phase of International Application No. PCT/AU2007/000556, entitled “DRESSING SECURING SYSTEM”, which was filed on Apr. 27, 2007, and which claims priority of Australian Patent Application No. 2006902179, filed Apr. 27, 2006.

### DESCRIPTION

### Field of the Disclosure

The disclosure relates to wound dressing and particularly to securing systems for securing a wound dressing relative to a wound.

### BACKGROUND OF THE DISCLOSURE

The cleansing and debriding of wounds and the removal of wound exudate is important to the process of healing wounds. Commonly used wound dressings comprise gauze, foams, sponges, cotton wads or other fibrous materials. It is critical to maintain a sterile environment around the wound and to secure the wound dressing relative to the wound.

### SUMMARY OF THE DISCLOSURE

Disclosed is a dressing securing system for securing a dressing relative to a wound, the dressing securing system comprising a fabric composed of a resilient knitted material.

In one form the resilient knitted material comprises a multi-fibre knitted material.

In one form the dressing securing system further comprises a moisture-resistant or waterproof inner layer.

In one form the fabric is impregnated with a composition that undergoes a physical change upon the application of heat to the composition.

In one form the fabric incorporates a zig-zag stitch such that the magnitude of the extension of the fabric can be gauged.

Further, disclosed is a method of securing a dressing relative to a wound comprising binding the dressing to the wound with a fabric composed of a resilient knitted material including an elastic yarn.

### BRIEF DESCRIPTION OF THE DRAWINGS

Embodiments of the dressing security system will now be described, by way of example only, with reference to the accompanying drawings in which:

FIG. 1 shows an inside view of the dressing securing system;

FIG. 2 shows a cross sectional view of the dressing securing system of FIG. 1 in use; and,

FIG. 3 shows the dressing securing system of FIG. 1 in use.

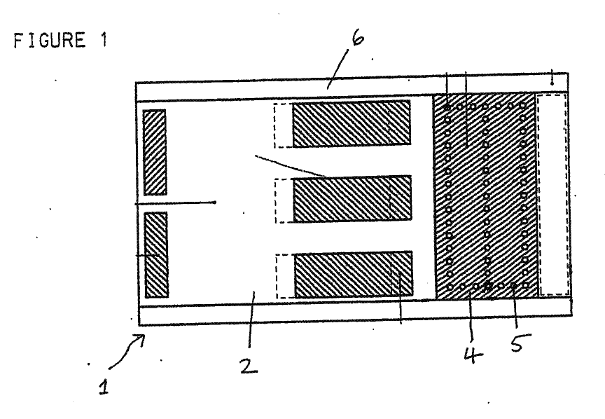

### DETAILED DESCRIPTION OF A PREFERRED EMBODIMENT

Referring to the figures, disclosed is a dressing securing system 1 for securing a dressing relative to a wound. The dressing securing system comprises a fabric 2 composed of a resilient knitted material. The resilient knitted material comprises a multi-fibre knitted material which may be composed of one or more of latex, elastene, nylon and spandex. The resilient knitted material incorporates an elastic yarn which is a textured stretch yarn of synthetic organic filaments, an elastomeric yarn or an elastic combination yarn.

The dressing securing system 1 further includes a moisture resistant or waterproof inner layer 4. The moisture resistant or waterproof inner layer includes a plurality of apertures 5 extending through the inner layer 4. The inner layer 4 is resilient and is made from latex rubber sheet cut to shape, with apertures 5 extending through the latex sheet to allow air circulation to the affected area. The inner lining 4 is stitched on to the resilient knitted material 2.

The dressing system further comprises a cuff 6 extending along the edges of the resilient knitted material 2. The cuff 6 prevents foreign matter from entering under the resilient knitted material 2 in use and is composed of a resilient or elasticised fabric.

The resilient knitted material 2 is non-frayable. The material is also non-roll in that when positioned with tension the material has no tendency to roll in on itself. Finally, the material is self-adhering by means of the texture of the material, a hooking or clawing nature of the material when meeting itself under lateral tension or by an associated tackiness. As a result the material can be wrapped about a body part and remain in place as shown in FIG. 3. The resilient material has a resilience of greater than 1.5 to 1 allowing the material to be stretched sufficiently to firmly dress the body part and self-adhere.

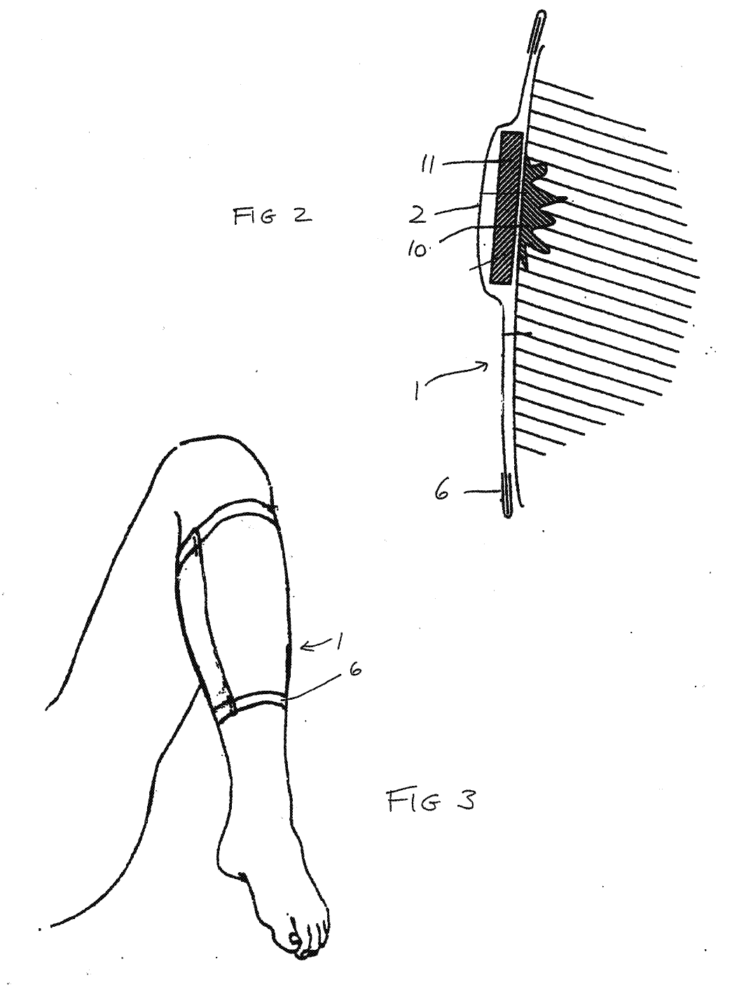

The dressing securing system is shown in use in FIGS. 2 and 3. In FIG. 2 the dressing securing system 1 is shown alongside a dressing 11 which is positioned adjacent to a wound 10. It can be seen that the dressing securing system 1 secures the dressing 11 in position over the wound 10.

The fabric of the dressing securing system 1 is impregnated with a polymer-based composition that undergoes a physical change upon the application of heat to the composition. In use, when the wound which is being dressed is transmitting excessive heat the fabric 2 of the dressing securing system 1 will undergo a physical change which is visible to the eye, for example the physical change is a colour change.

The fabric further incorporates a zig-zag stitch which is designed such that the magnitude of the extension of the fabric 2 can be gauged by the eye. In this regard the stitch can have a different colour or texture than the fabrica. Thus, if the anatomical part of the patient which is wounded undergoes swelling this will be visible from outside the dressing securing system. Alternately the fabric 2 can incorporate a polymer or other composition that undergoes a physical change such as a colour change upon extension of the material.

The dressing securing system 1 allows a patient or healthcare worker to easily dress a wound without applying a dressing too tightly or too loosely. The system 1 is sized to particularly fit a body part or guiding lines can be drawn or stitched to the system 1. Alternatively the zig-zag stitch can be utilised to gauge the tightness of the system 1. As applying a bandage too tightly can cause amputation or nerve damage and applying a bandage loosely can result in dislodgement of the dressing and infection this is vital to good wound care.

In the preceding description and in the claims which follow, except where the context requires otherwise due to express language or necessary implication, the word “comprise” or variations such as “comprises” or “comprising” is used in an inclusive sense, ie. to specify the presence of the stated features but not to preclude the presence or addition of further features in various embodiments.

Variations and modifications can be made in respect of the system described above and defined in the following statements of claim.

1. A dressing securing system for securing a dressing relative to a wound, the dressing securing system comprising a fabric composed of a resilient knitted material.

2. A dressing securing system as defined in claim 1, wherein the resilient knitted material comprises a multi-fibre knitted material.

3. A dressing securing system as defined in claim 1, wherein the resilient knitted material is composed of one or more of latex, elastene, nylon and spandex.

4. A dressing securing system as defined claim 1, wherein the resilient knitted material includes an elastic yarn which is a textured stretch yarn of synthetic organic filaments, an elastomeric yarn or an elastic combination yarn.

5. A dressing securing system as defined in claim 1, further comprising a moisture-resistant or waterproof inner layer.

6. A dressing securing system as defined in claim 5, wherein the inner layer has a plurality of apertures extending therethrough.

7. A dressing securing system as defined in claim 6, wherein the inner layer is resilient.

8. A dressing securing system as defined in claim 1, wherein the fabric has a resilience of more than 1.5 to 1.

9. A dressing securing system as defined in claim 8, wherein the fabric is non-frayable.

10. A dressing securing system as defined in claim 9, wherein the fabric is non-roll.

11. A dressing securing system as defined in claim 10, wherein the fabric is self-adhering.

12. A dressing securing system as defined in claim 1, wherein the fabric is impregnated with a composition that undergoes a physical change upon the application of heat to the composition.

13. A dressing securing system as defined in claim 12, wherein the physical change is a colour change.

14. A dressing securing system as defined in claim 1, wherein the fabric incorporates a zig-zag stitch oriented such that the magnitude of the extension of the fabric can be gauged.

15. A method of securing a dressing relative to a wound comprising binding the dressing to the wound with a fabric composed of a resilient knitted material including an elastic yarn.

16. A method of securing a dressing as defined in claim 15, wherein the elastic yarn of the knitted fabric is a textured stretch yarn of synthetic organic filaments, an elastomeric yarn or an elastic combination yarn.

17. A method of securing a dressing as defined in claim 15, wherein the resilient knitted material comprises a multi-fibre knitted material.

18. A method of securing a dressing as defined in claim 15, wherein the resilient knitted material is composed of one or more of latex, elastene, nylon and spandex.

19. A method of securing a dressing as defined in claim 15, wherein the fabric has a moisture-resistant or waterproof inner layer.

20. A method of securing a dressing as defined in claim 19, wherein the inner layer has a plurality of apertures extending therethrough.

21. A method of securing a dressing as defined in claim 19, wherein the inner layer is resilient.

22. A method of securing a dressing as defined in claim 15, wherein the fabric has a resilience of more than 1.5 to 1.

23.-28. (canceled)
